(19) United States
(12) Patent Application Publication
(10) Pub. No.: US 2005/0154458 A1
(43) Pub. Date: Jul. 14, 2005

(54) Apparatus for the regeneration of human tissue

(75) Inventor: Matthias Steinwachs, Freiburg (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica International S. DE R.L., Limerick (IE)

(21) Appl. No.: 10/945,207

(22) Filed: Sep. 20, 2004

(30) Foreign Application Priority Data
Sep. 22, 2003 (GB) 03 22145.4

(51) Int. Cl. A61F 2/02

(52) U.S. Cl. 623/11.11; 606/151

(57) ABSTRACT

An apparatus for use in the regeneration of structured human tissue has a pair of opposed surfaces comprising a bio material or a synthetic polymer material. Each surface carries an active bio layer which can interact with stem cells from bone marrow. The opposed surfaces can be moved from an open position where they are spaced apart to a closed position in which they are closer together to form a multi-layer or sandwich construction.

### BACKGROUND OF THE INVENTION

This application relates to an apparatus for use in the regeneration of structured human tissue and is particularly, although not exclusively, applicable for meniscus, disc, tendon and ligament applications.

### SUMMARY OF THE INVENTION

According to the present invention, an apparatus for use in the regeneration of structured human tissue comprises a pair of opposed surfaces of bio material or a synthetic polymer material, each surface carrying an active bio layer which can interact with stem cells from bone marrow, and which can be moved from an open position where the opposed surfaces are spaced apart to a closed position in which they are closer together to form a multi-layer or sandwich construction.

The bio material or synthetic polymer material is preferably flexible and in a preferred embodiment the opposed surfaces are provided on a folded sheet. In another convenient construction the opposed surfaces can be provided on the inner surfaces of a tube.

A device can be provided for holding the surfaces in the closed position, for example by integrated sutures, filaments or bands.

A device can also be provided for holding the surfaces in position on a bone or other human organ for example a meniscus, disc, tendon or ligament.

In a preferred embodiment, the bio material is of Collagen I type and the active bio layer is Hyaluronic acid. Alternately, the bio material may be a combination of a natural and synthetic polymer with an active bio layer.

The synthetic polymer material can be a polyvinyl alcohol, acetate hydrogel, polycaprolactone, polyurethane, alpha and beta hydroxy acids or esters and combinations thereof, or other suitable porous or biogradable polymers that can be fabricated in the form of a scaffold or matrix to support the active bio layer.

The construction of the apparatus is such that it enables the active bio layer to be impregnated with stem cells from bone marrow taken from the patient and prior to closing the surfaces together. The advantage is that even in a deep bio layer a homogenous distribution of the stem cells in the form of bone marrow blood can be achieved. With existing techniques this is not possible. Additionally, it enables an impregnation of any other cells, growth factors, cytokines, coating materials, and any other bioactive materials.

### BRIEF DESCRIPTION OF THE DRAWINGS

The invention can be performed in various ways and some embodiments will now be described by way of example and with reference to the accompanying drawings in which:

FIG. 1 is a pictorial oblique view of the apparatus according to the invention ready for use;

FIG. 2 shows how filtrated human bone marrow (blood) can be applied to the opened surfaces;

FIG. 3 shows the surfaces according to the apparatus in the closed position and ready for use;

FIG. 4 is a pictorial representation of a tube embodying the invention;

FIG. 5 shows the tube in a closed position;

FIG. 6 shows how the opposed surfaces of bio material or synthetic polymer material can be provided as two separate elements; and

FIG. 7 is a pictorial oblique view showing the overall shape of another construction according to the invention.

### DETAILED DESCRIPTION

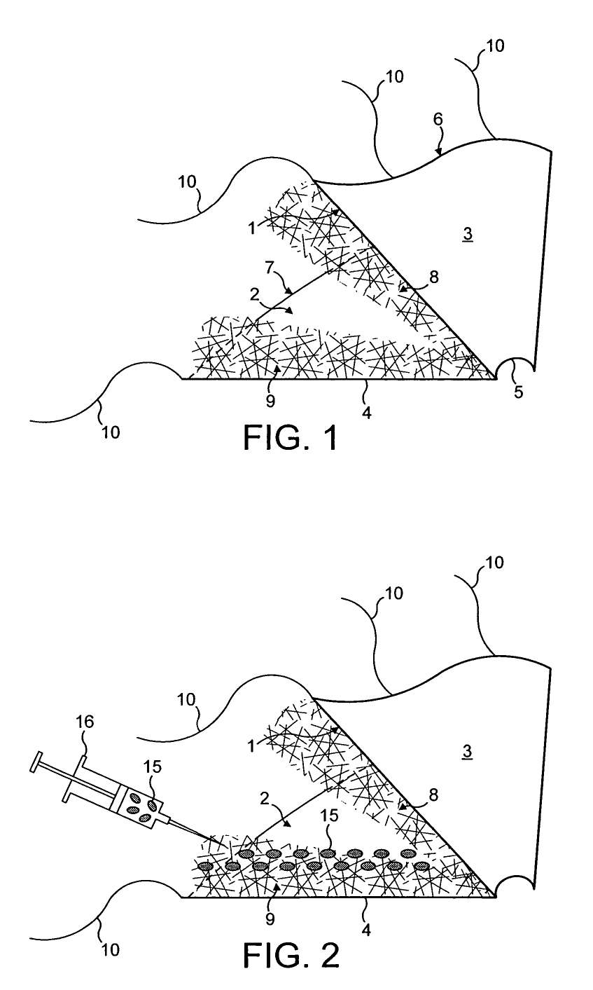

As shown in FIGS. 1 to 3 the apparatus for use in the regeneration of structured human tissue according to the present invention comprises a pair of opposed surfaces 1 and 2 which are formed on a sheet or film of bio material, for example Collagen I or synthetic polymers and other resorbable materials. The sheet has a double triangular wedge configuration, surface 1 being on a first generally triangular shaped sheet portion 3 and surface 2 on a second triangular sheet portion 4. The adjoining ends of the two generally triangular portions 3 and 4 are indicated by joining portion 5 and the opposing sides are indicated by reference numerals 6 and 7. The surfaces 1 and 2 each carry an active bio layer indicated by reference numerals 8 and 9. In order to make the drawing more clear only the edges of the bio layer are shown on each surface. The bio material or synthetic polymer material is flexible.

Ties in the form of stitches or bands 10 are provided so that the surfaces can be held together in a closed position.

FIG. 2 shows how the stem cells, cytokines, growth factors, other cells, harvested from bone marrow, indicated by reference numeral 15, in a syringe 16, can be applied to one of the active bio layers 9. The stem cells are provided by filtrated human bone marrow in the form of bone marrow blood taken from the patient. This can be done by creating a drill hole into an appropriate bone, aspirating the bone marrow blood and filtrating it.

With the apparatus according the invention even in a deep bio layer a homogenous distribution of the blood solution can be achieved.

The blood solution will include cells of (hMSC fibrocytes, chondrocytes) and proteins (cytokines, growth factors).

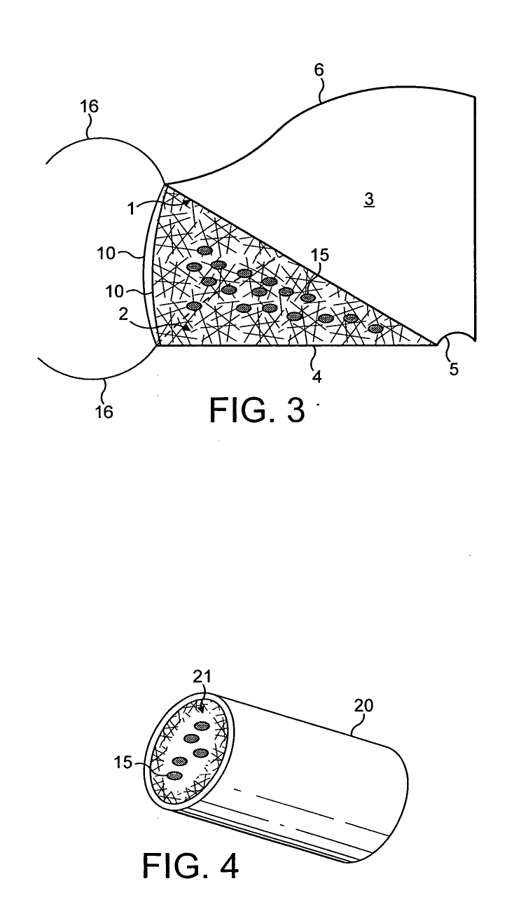

FIG. 3 shows how the surfaces 1 and 2 can be folded together to a closed position and can be held in place by the ties 10. The ties 10 can be in the form of stitches or bands which are integrated into the material.

Additionally, bands of the synovia which are prepared during surgery can be used for fixation purposes. The synovia laps can surround the construct or just cover a surface.

Additional anchoring ties 16 can be provided as shown in FIG. 3 and these are used to attach the apparatus to the appropriate bone or human organ, for example a meniscus, disc, tendon or ligament.

As will be seen from the drawings, a sandwich construction is achieved.

When installed the apparatus will be partially surrounded by synovia and fibrochondrocytes, hMSC's and growth factors and cytokines an other cells from the bone marrow blood or external coating together with local blood vessels will all assist in the regeneration of structured human tissue which is required.

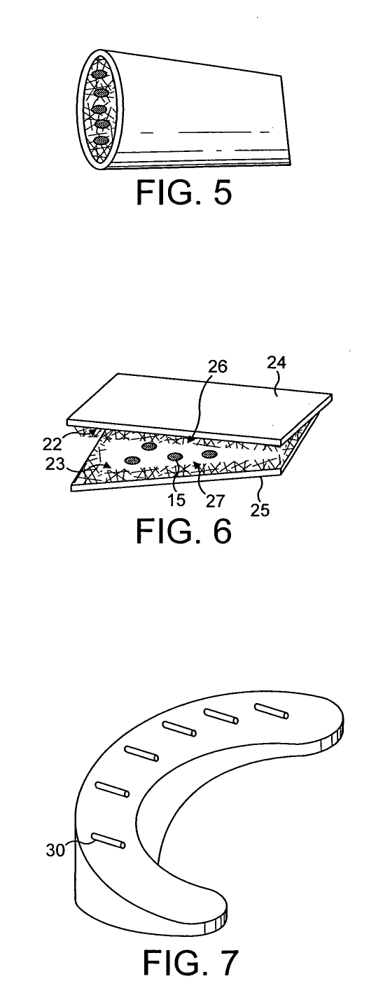

FIG. 4 shows an alternative construction for providing a pair of opposed surfaces of bio material or synthetic polymer material which comprises a tube 20 of the appropriate material and the inner surface of which carries an active bio layer 21. As in the previous figures, only the edge of the bio layer is shown in the drawing. The filtrated human bone marrow 15 is applied onto the active bio layer 21 while the tube is in the open position shown in FIG. 4. The tube can now be moved to a closed position, as shown in FIG. 5, where a sandwich construction is again achieved. Ties 10 and 16 can again be provided but are not shown in the drawing.

In another alternative construction, as shown in FIG. 6, the surfaces 22 and 23 are formed on two flat sheets 24 and 25 of bio material or synthetic polymer material. Active bio layers 26 and 27 are provided on the sheets. As in the previous embodiments only the edges of the layers are shown. Filtrated human bone marrow or other cells, cytokines, growth factors or coating materials can again be injected onto the bio layer 27 and the sheets closed together by ties 10 (not shown). Ties 16 can also be included to hold the surfaces in the closed position.

FIG. 7 is a pictorial oblique view of the overall general shape of another construction according to the invention. The overall shape is of a crescent shaped meniscus which can be fitted in position in the manner shown in FIGS. 1, 2 and 3. This figure also shows how the bio material or synthetic polymeric material can carry a number of tubes, indicated by reference numeral 30. These tubes will allow, by capillary action the injection of the cells to be distributed within the material. Tubes of this kind can also be employed in any of the constructions shown in the other figures.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An apparatus for use in the regeneration of structured human tissue comprising a pair of opposed surfaces of bio material or a synthetic polymer material, each surface carrying an active bio layer which can interact with stem cells from bone marrow, and which can be moved from an open position where the opposed surfaces are spaced apart to a closed position in which the opposed surfaces are closer together to form a multi-layer or sandwich construction.

2. The apparatus as set forth in claim 1 wherein said opposed surfaces are provided on a folded sheet.

3. The apparatus as set forth in claim 1 wherein said opposed surfaces are provided on the inner surfaces of a tube.

4. The apparatus as set forth in claim 1 further comprising means for holding the surfaces in the closed position.

5. The apparatus as set forth in claim 1 further comprising means for holding the surfaces in position on a bone, or other human organ.

6. The apparatus as set forth in claim 5 wherein the human organ is a meniscus, disc, tendon or ligament.

7. The apparatus as set forth in claim 1 wherein the bio material is of Collagen I type and the active bio layer is Hyaluronic acid.

8. The apparatus as set forth in claim 1 wherein the bio material is a combination of a natural and synthetic polymer with an active bio layer.

9. The apparatus as set forth in claim 1 wherein the synthetic polymer material is selected from the group consisting of polyvinyl alcohol, acetate hydrogel, polycaprolactone, polyurethane, alpha and beta hydroxy acids or esters and combinations thereof, or other suitable porous or biodegradable polymers that can be fabricated in the form of a scaffold or matrix to support the active bio layer.

10. A method for forming a tissue scaffold comprising:
providing a generally flat sheet of biocompatible material;
applying a material containing hyaluronic acid to the sheet;
applying stem cells to the hyaluronic acid coated sheet;
folding the coated sheet to form a closed structure;
holding the closed structure in the closed position by using of tying elements; and
attaching the closed sheet to tissue.

11. The method as set forth in claim 10 wherein the sheet is made from type I Collagen.

12. The method as set forth in claim 11 wherein the sheet is made of a member selected from the group consisting of polyvinyl alcohol, acetate hydrogel, polycaprolactone, polyurethane, alpha and beta hydroxy acids or esters and combinations thereof, or other suitable porous or biodegradable polymers that can be fabricated in the form of a scaffold or matrix to support the hyaluronic acid.
